(19) United States
(12) Patent Application Publication
(10) Pub. No.: US 2004/0266858 A1
(43) Pub. Date: Dec. 30, 2004

(54) Stable, acid, aqueous solution containing alpha-liponic acid (derivatives), method for the production thereof and use of the same

(76) Inventors: Hans Schuhbauer, Edmonton (CA); Stephan Winkler, Obing (DE); Margot Rudholzner, Trostberg (DE); Roswitha Mussner, Trostberg (DE); Evi Huber, Garching (DE)

Correspondence Address: FULBRIGHT & JAWORSKI, LLP, 666 FIFTH AVE, NEW YORK, NY, 10103-3198, US

(21) Appl. No.: 10/495,600

(22) PCT Filed: Nov. 28, 2002
(86) PCT No.: PCT/EP02/13446

(30) Foreign Application Priority Data
Dec. 3, 2001 (DE) 101 59 245.0

(51) Int. Cl. A61K 31/385

(52) U.S. Cl. 514/440

(57) ABSTRACT

The present invention describes a stable, acidic, aqueous solution of α-lipoic acid or derivatives thereof. A content of the lipoic acid component employed which is above the intrinsic solubility thereof in the aqueous solution is achieved preferably by a production method in which in a first stage a stock solution of the lipoic acid component in water is provided with the aid of a base, then this stock solution is brought where appropriate by dilution and temperature control into the desired concentration range and temperature range, and finally the desired pH of the solution is adjusted by adding an acid. The solution obtained inter alia in this way is, owing to its stable properties, outstandingly suitable for use for pharmaceutical, cosmetic, dietary or else technical purposes, for example for diminishing photochemical effects.

The present application relates to a stable, acidic, aqueous solution containing α-lipoic acid (derivatives), a method for the production thereof and the use of the same.

α-Lipoic acid (thioctic acid, 1,2-dithiolane-3-pentanoic acid) has been known for about 50 years as a growth factor in microorganisms, but it also occurs as the R-(+) enantiomer in low concentrations in higher plants and animals. The physiological action of α-lipoic acid in hydrophilic and lipophilic media is as coenzyme of the oxidative decarboxylation of α-keto carboxylic acids (e.g. pyruvic acid, α-ketoglutaric acid). In addition, α-lipoic acid is also involved as cofactor in the degradation of certain amino acids. It moreover contributes to the regeneration of vitamin C, vitamin E, glutathione and coenzyme Q10. However, α-lipoic acid and its relevant redox partner dihydrolipoic acid also have strongly antioxidant and occasionally also prooxidant properties; α-lipoic acid is therefore often referred to as “universal antioxidant”.

Racemic α-lipoic acid is approved for the treatment of liver disorders and neuropathies (e.g. diabetic polyneuropathy); its use as an efficient inhibitor of the replication of HIV-1 viruses has been suggested (cf. Klin. Wochenschr. 1991, 69(15), 722-724). The R enantiomer of α-lipoic acid is in clinical phase II in Germany (since December 2000) and the USA (since May 2001). Racemic α-lipoic acid is employed both as pure solid mixed with other components, in solid pharmaceutical formulations and in ampules or capsules, but also in infusion solutions, as active pharmaceutical ingredient or as food supplement. Solutions for injection of α-lipoic acid are preferably employed mainly in the initial stage of a corresponding clinical therapy.

A considerable disadvantage of racemic or enantiopure α-lipoic acid is its instability toward light and temperature, and a general tendency to polymerizability. The reason for this derives from the extreme ease of cleavability of the characteristic disulfide linkage of the strained five-membered ring in the lipophilic part of the molecule. This cleavage brings about an intermolecular formation of disulfide bridges, leading to dimeric, oligomeric and polymeric lipoic acid derivatives (DE patent 16 17 740). This may take place under the influence of light or temperature, but also through the addition of suitable nucleophiles (J. Org. Chem. 1969, 34, 3131). Decomposition by oxidation is also known from the literature (J. Org. Chem. 1975, 40, 58-62). The polymerizability of the pure enantiomers of (α-lipoic acid is even more pronounced than that of the racemate. In addition, the low acidity and high lipophilicity of α-lipoic acid mean that the solubility in water is extremely low at a neutral, physiological or acidic pH (table 1):

TABLE 1
Solubility of (±)-α-lipoic acid in water

| Temperature | 4° C. | 8° C. | 12° C. | 20° C. | 30° C. | 40° C. |
|---|---|---|---|---|---|---|
| α-Lipoic acid | 0.025 | 0.04 | 0.045 | 0.08 | 0.11 | 0.20 |
| [% by wt] | | | | | | |

The concentrations and absolute contents, which are limited by this intrinsic solubility, of α-lipoic acid in water at neutral, physiological or acidic pH are thus far below the effective doses necessary for pharmaceutical, dietary and cosmetic applications.

In pharmaceutical technology, therefore, salts of α-lipoic acid are normally used because, as a weak organic acid, the solubility of (α-lipoic acid is very much higher in the alkaline range. However, aqueous alkaline solutions of α-lipoic acid with various salt formers show a considerable tendency to spontaneous and/or continued turbidity or precipitation. Since from the regulatory viewpoint solutions for injection which show a tendency to turbidity or precipitation during their storage are regarded as “decomposing” or “decomposed”, they are no longer allowed to be used pharmaceutically.

Various solutions for injection produced by reacting α-lipoic acid with appropriate salt formers also lead when used pharmaceutically to a number of complications and intolerances, e.g. somatoform stress, episodes of skin reddening with feeling of warmth, respiratory symptoms or headaches. Thus, for example, it is known that an increase in the ethylenediamine content in corresponding lipoic acid salts makes the tolerability worse.

In addition, the physiological, neutral or weakly alkali nature of the aqueous lipoic acid solutions known in the prior art restricts the applicability thereof. The aqueous solutions of the lipoic acid salt with trometamol which were prepared in EP 318 891 B1 show a basic pH of from 7.6 to 8.8. This patent quotes French and Spanish patents according to which 1% strength aqueous solutions are prepared by an equimolar reaction of α-lipoic acid with amino acids resulting in a neutral pH for these solutions. German published specifications 10 47 991 and 10 56 784 disclose injectable solutions of lipoic acid salts which have a physiological or weakly alkaline pH. In Pharmazie 1988, 41, 98, commercially available lipoic acid solutions for infusion (e.g. Hepasteril® B compositum forte, Sterofundin® CH compositum, Tutofusin® LC forte, Thioctacid®) with pH values of from 6.64 to 8.35 were subjected to a stability test.

The use of α-lipoic acid for example in physiologically effective concentrations as food supplement in beverages having an acidic pH of from 2.5 to 4 has not to date been possible.

It was thus an object of the present invention to provide a stable, acidic, aqueous solution containing α-lipoic acid or derivatives thereof which contains the lipoic acid component both in physiologically effective concentrations and ensures a generally good tolerability and broad applicability.

This object has been achieved with an appropriate solution which contains the lipoic acid component, based on the total weight of the solution, in proportions between 0.001 and 30% by weight and which have a pH between 0.0 and 6.5.

It has surprisingly been found that the aqueous solutions of the invention can have considerably higher lipoic acid contents than would have been expected from the conventional solubility of α-lipoic acid in water (cf. table 1). It was likewise not possible to expect that neither turbidity nor precipitation of a solid occur even on continued storage of the claimed solution, preferably for a period of at least one year at a temperature of 15 to 40° C. and in a pH range from 2.5 to 4.0. On the contrary, the solutions of the invention remain clear and without any precipitate over the entire storage period, resulting in further advantages in relation to the universality of the possible applications in the area of pharmaceutical formulations and pharmaceutical, dietary or cosmetic applications.

Suitable as preferred lipoic acid component for the aqueous solutions of the invention are racemic α-lipoic acid, enantiopure R-(+)- or S-(−)-α-lipoic acid or any mixtures thereof, as well as racemic dihydrolipoic acid (6,8-dimercaptooctanoic acid), enantiopure R-(−)- or S-(+)-dihydrolipoic acid or any mixtures thereof. A further possibility provided is the use of α-lipoic acid or dihydrolipoic acid as such or wholly or partly in the form of their salts such as, for example, creatine, sodium, potassium, ammonium or ornithine lipoates for the production of the solution. The production of racemic α-lipoic acid, of enantiopure or enantiomer-enriched R-(+)- or S-(−)-α-lipoic acid, of racemic dihydrolipoic acid, enantiopure or enantiomer-enriched R-(−)- or S-(+)-dihydrolipoic acid and of salts or mixtures thereof can take place in a known manner.

The proportions from the broad range, which is essential according to the invention, of lipoic acid contents are, in each case based on the total weight of the aqueous solution of the invention, preferably between 0.01 and 10% by weight and particularly preferably between 0.2 and 5% by weight. All proportions by weight stated herein are based on racemic or optically pure α-lipoic acid. This means that on use of lipoic acid derivatives or salts the amounts stated in each case for the dosages correspond to those of free lipoic acid and thus must be adapted to the altered molecular weight. No loss of α-lipoic acid through decomposition or polymerization is found within the scope of the invention if the pH values of the solutions vary within the range which is likewise essential according to the invention and from which values between 2.0 and 5.5 and in particular between 2.5 and 4.0 are to be regarded as preferred.

It is finally also provided for the solution of the invention to contain in certain cases where appropriate further conventional pharmaceutical excipients and/or formulation aids such as, for example, ethanol, liquid polyethylene glycols (PEG), in particular of the 200-600 types, propylene glycol, butylene glycol, tetraglycol, benzyl alcohol, sorbitol, mannitol or glycerol.

Sterilization of the solution of the invention which may be necessary, for example with superheated steam, is likewise possible in principle. Before being dispensed, for example into ampules or beverage cans, the solutions of the invention can also if required be filtered, for example through a blue ribbon or black ribbon filter.

Besides the solution itself and its variants, the present invention also claims a method for its production, in which

(a) firstly an aqueous stock solution of the lipoic acid component with a pH between 5.5 and 14.0 is prepared with the aid of a base, then

(b) where appropriate the content of the lipoic acid component in the stock solution obtained from stage (a) is adjusted by dilution to from 0.001 to 30% by weight and/or the temperature is brought to values between −5 and 80° C. by temperature control, and finally

(c) the pH of the solution from stage (b) is adjusted to values between 0.0 and 6.5 with the aid of an acid.

The stock solution is produced in stage (a) by adding α-lipoic acid or a suitable derivative and a likewise suitable base to the required amount of water. The sequence of the addition of the individual component can be varied as desired. However, the addition should take place in all possible stages (a), (b) and (c) at temperatures of from −5 to 80° C., preferably from 0 to 50° C. and particularly preferably from 4 to 30° C.

The temperature of the respective solutions between the individual steps in the method of the procedure of the invention can moreover be controlled as desired within the stated wide temperature limits.

Suitable for producing the stock solution of α-lipoic acid or derivatives thereof are, in particular, Brönsted and/or Lewis bases which contain cationic components from the series of alkali metals (such as, for example, sodium or potassium) or alkaline earth metals (such as, for example, calcium or magnesium). The hydroxides, thiolates, acetates, carbonates and hydrogencarbonates are particularly suitable as anion. However, it is also possible without difficulty to have recourse to other bases, in which case the cationic component thereof ought according to the invention to be derived in particular from the series of iron, copper, zinc, palladium, vanadium and selenium. Bases which are also extremely suitable for the aqueous solutions of the present invention contain organic cations and, in this connection, preferably open-chain or cyclic ammonium compounds such as benzylammonium, diisopropylammonium, triethylammonium or cyclohexyl-ammonium, or complex cations where appropriate with metallic central atoms such as, for example, iron(III), chromium(III) or cobalt(II) and/or neutral, cationic or anionic ligands such as, for example, water, ammonia, carbonyl, cyano or nitroso, or oxo cations such as oxovanadium(V) (VO³⁺) or oxovanadium(IV) (VO²⁺)

It is not generally necessary to produce the stock solutions through equimolar proportions of the lipoic acid component and the appropriate base for producing the aqueous solutions of the invention. On the contrary, it has emerged that stock solutions with asymmetrical stoichiometries also lead to the stable, acidic, aqueous solutions of the invention with a clear appearance and tolerable properties. This is because the crucial criterion for this is evidently not the stoichiometry between the lipoic acid component, the base and the acid, but the attainment of a clear stock solution in stage (a). Thus, following the present invention, for example a first solution containing 208 g of (±)-dihydrolipoic acid (1.0 mol) with 64 g of a 50% strength aqueous sodium hydroxide solution (0.8 mol) in 1.9 l of water yields a clear solution which can be adjusted to a pH of between 2.0 to 6.5 by addition of acid. Verification of the clarity of this solution results from inspection, but it can also be undertaken by conventional measurements, familiar to the skilled worker, of turbidity relative to standard solutions.

It is possible in general to employ the base component in stage (a) in amounts of from 0.1 to 5.0 mole equivalents, preferably from 0.3 to 3.0 mole equivalents and particularly preferably from 0.6 to 1.5 mole equivalents, in each case based on α-lipoic acid.

It is likewise to be regarded overall as preferred for the pH at the end of stage (a) to be from 6.0 to 11.0 and particularly preferably between 6.5 and 10.5.

In a preferred embodiment of the present invention, the appropriate stock solution of α-lipoic acid, a derivative or a suitable precursor is produced in stage (a) in concentrated form and diluted before the acidification in stage (c) in stage (b) to the desired concentration range, which preferably takes place with water.

Acids which can be employed within the scope of the present invention in stage (c) are, in particular, physiologically tolerated acids, e.g. organic or inorganic Brönsted acids such as hydrochloric acid, acetic acid, ascorbic acid and glutamic acid, as well as organic or inorganic Lewis acids, from the series of which in particular carbon dioxide, Ca²⁺ and Fe²⁺ are particularly suitable. However, also suitable are complex acids, in particular hexaaquoaluminum(III) [Al(H₂O)₆³⁺], and polymeric acids, of which. polyphosphoric acid (PPA), an isopolyacid such as, for example, heptamolybdic acid (H₆Mo₇O₂₄) or a heteropolyacid such as, for example, dodecatungstophosphoric acid (H₃[PW₁₂O₄₀]) are particularly to be preferred. Finally, it is also possible in this connection to use any mixtures of individual acid forms among one another or else between the individual acid forms. The acid forms or mixtures thereof can preferably be employed in amounts of from 0.1 to 5.0 mole equivalents, preferably from 0.2 to 4.0 mole equivalents and particularly preferably from 0.9 to 2.0 mole equivalents, in each case based on α-lipoic acid.

Finally, the present invention also takes account of the use of the solution of the invention for pharmaceutical, cosmetic or dietary purposes, in particular within the framework of a supplementary or combination therapy or else as solution for infusion.

It is particularly preferred in this connection for the stable, acidic, aqueous solution of the present invention to be used in beverages which have, in particular, a pH of from 2.0 to 5.5, preferably between 2.5 and 4.0 and particularly preferably between 3.0 and 3.5.

In a specific variant of the use, the solution of the invention is employed to prevent or diminish photochemical damage and, in this regard, especially for that caused by insolation, UVA radiation, UVB radiation, X-radiation, gamma radiation and mixtures thereof, and which occur particularly preferably in humans or animals.

The solution of the invention can also be employed as fiber-treatment composition, in which case it acts in particular on the respective fiber material and subsequently the solvent used is removed particularly preferably by drying or spinning. Fibers taken into account for this application are those in particular composed of cotton, linen, live wool, wool, natural silk, keratin, synthetic fibers or any mixtures thereof.

The last-mentioned particular purpose of use in connection with photochemical damage in particular covers an especially advantageous wide range because, besides the traditional pharmaceutical and cosmetic areas of application and areas of food and nutrition industry, for example also biotechnological areas of application come under consideration, for example through the addition of the solution of the invention to culture media.

However, a purely technological application is also conceivable, for example in the form of an additive to lubricant oils or as fiber-treatment composition in its specific form as textile- or hair-treatment composition, in which case it is suitable in particular as sunscreen composition which is then not directly applied to the skin or else preserves natural or artificial hair from the destructive effects of radiation.

Overall, the present invention offers, through the provision of the claimed stable, acidic, aqueous solution containing lipoic acid components, a convincing further development of prior art, which is positively supplemented in particular through the additionally claimed and relatively simple production method.

The following examples illustrate these advantages of the present invention.

### EXAMPLES

### Example 1

1.0 g of α-lipoic acid were added at room temperature to a solution of 0.24 g of sodium hydroxide in 98.76 g of water, a pH of 11.5 being set up. The solution was controlled to a temperature of 40° C. and stirred for 30 min, and was then acidified with 15% strength aqueous HCl to a pH of 5.9. Determination of the content by HPLC revealed an α-lipoic acid content of 1.07±0.1% by weight in the solution.

### Example 2

480 g of 50% strength aqueous sodium hydroxide solution were added to a suspension of 1000 g of α-lipoic acid in 198.52 kg of water at room temperature and stirred for 60 min. A pH of 11.65 was set up during this. The clear solution was controlled to a temperature of 40° C. and stirred for a further 30 min. Half-concentrated hydrochloric acid was then used to adjust to a pH of 5.3. Determination of the content by HPLC revealed an α-lipoic acid content of 0.55±0.1% by weight in the solution.

### Example 3

2.0 kg of α-lipoic acid and 0.47 kg of solid sodium hydroxide were added at 40° C. to 97.53 kg of water. The resulting solution was stirred for 60 min, during which a pH of 12.0 was set up. The solution was then acidified with dilute hydrochloric acid to a pH of 6.2. Determination of the content by HPLC revealed an α-lipoic acid content of 2.14±0.15% by weight in the solution.

### Example 4

5 g of α-lipoic acid were added at room temperature to a solution of 10 g of 50% strength aqueous potassium hydroxide solution in 9985 g of water. The clear solution was stirred for 30 min, during which a pH of 12.96 was set up. The solution was cooled to 4° C. and acidified with 15% strength hydrochloric acid to a pH of 2.0. Determination of the content revealed an α-lipoic acid content of 0.06±0.03% by weight in the solution.

### Example 5

10 g of R-(+)-α-lipoic acid were added at room temperature to a solution of 10 g of potassium hydroxide in 9980 g of water. The clear solution was stirred for 30 min, during which a pH of 13.0 was set up. The solution was cooled to 4° C. and acidified with 15% strength hydrochloric acid to a pH of 2.1. Determination of the content revealed an R-(+)-α-lipoic acid content of 0.11±0.04% by weight in the solution.

### Example 6

208 g of (±)-dihydrolipoic acid were added to 64 g of a 50% strength aqueous sodium hydroxide solution in 1.9 l of water. The clear solution was stirred at room temperature for 30 min, the temperature was controlled to 20° C., and then 10% strength hydrochloric acid was used to acidify to a pH of 3.0 at 20° C. The solution remained clear. Determination of the content by HPLC revealed a dihydrolipoic acid content of 0.90% by weight in the solution.

1-29. (canceled)

30. An aqueous solution comprising water and an α-lipoic acid compound comprising between 0.001 to 30% by weight of α-lipoic acid or a derivative thereof based on the total weight of the solution and leaving a pH of between 0.0 and 6.5.

31. The solution as claimed in claim 30, wherein said α-lipoic acid or derivative thereof is selected from the group consisting of a racemic α-lipoic acid, an enantiopure R-(+)-α-lipoic, an enantiopure S-(−)-α-lipoic acid and mixtures thereof.

32. The solution as claimed in claim 30, wherein said α-lipoic acid or derivative thereof is selected from the group consisting of a racemic dihydrolipoic acid, an enantiopure R-(−)-α-hydrolipoic acid, an enantiopure S-(+)-dihydrolipoic acid, and mixtures thereof.

33. The solution as claimed in claim 31, further comprising a racemic dihydrolipoic acid, an enantiopure R-(−)-α-lipoic dihydrolipoic acid, an enantiopure S-(+)-dihydrolipoic acid, a mixture thereof.

34. The solution as claimed in claim 30, wherein the α-lipoic acid or derivative thereof comprises a salt.

35. The solution as claimed in claim 30, wherein the proportion of he lipoic acid or derivative thereof is between 0.01 and 10.0% by weight based on the total weight of the solution.

36. The solution as claimed in claim 30, having a pH of between 2.0 and 5.5.

37. The solution as claimed in claim 30, further comprising at least one of a pharmaceutical excipient or formulation aid.

38. A method comprising
(a) preparing an aqueous stock solution of a an α-lipoic acid component comprising α-lipoic acid or a derivative thereof with a pH between 5.5 and 14.0 by adding a base;
(b) adjusting the content of the α-lipoic acid component in the stock solution obtained from step by dilution to from 0.001 to 30% by weight or by adjusting the temperature to between −5 and 80° C. by temperature control, or both, and
(c) adjusting the pH of the solution. from stage (b) to a value between 0.0 and 6.5 by adding an acid to produce a stable, acid, aqueous solution comprising water and an α-lipoic acid component comprising α-lipoic acid or a derivative thereof comprising the lipoic acid component, based on the total weight of the solution, in proportions between 0.001 and 30% by weight and has a pH of between 0.0 and 6.5.

39. The method as claimed in claim 38, wherein said base is a Brönsted base, a Lewis base or a combination thereof.

40. The method as claimed in claim 38, wherein said base comprises a cation of alkali metals or alkaline earth metals.

41. The method as claimed in claim 39, wherein said base comprises a cation of alkali metals or alkaline earth metals.

42. The method as claimed in claim 38, wherein said base comprises a cation selected from the group consisting of iron, copper, zinc, palladium, vanadium and selenium.

43. The method as claimed in claim 38, wherein said base comprises an organic cations.

44. The method as claimed in claim 38, wherein said base is employed in an amount of from 0.1 to 5.0 mole equivalents based on the α-lipoic acid component.

45. The method as claimed in claim 38, wherein the temperature in step (a) is from −5 to 80° C.

46. The method as claimed in claim 38, wherein the pH of the solution at the end of step (a) is from 6.0 to 11.0.

47. The method as claimed in claim 38, wherein the stock solution from step (a) is diluted in step (b) with water.

48. The method as claimed in claim 38, wherein the stock solution from step (a) is brought in step (b) before the dilution or the optionally diluted stock solution in stage (b) to a temperature in the range from −5 to 80° C.

49. The method as claimed in claim 38, wherein in stage (c) said acid is an organic or inorganic Brönsted acid.

50. The method as claimed in claim 38, wherein in stage (c) said acid is an organic or inorganic Lewis acid.

51. The method as claimed in claim 38, wherein in stage (c) said acid is a complex acid.

52. The method as claimed in claim 38, wherein in stage (c) said acid is selected from the group consisting of a polymeric acid, an isopolyacid, and a heteropolyacid.

53. The method as claimed in claim 38, wherein that the acid is employed in stage (c) in an amount of from 0.1 to 5.0 mole equivalent based on the lipoic acid component.

54. A method comprising administering a suitable amount of the solution of claim 30 to a subject in need of supplementary or combination therapy.

55. A beverage comprising the solution as claimed in claims 30.

56. A method for preventing or diminishing photochemical damage caused by insolation, UVA radiation, UVB radiation or X-radiation as gamma radiation in humans or animals comprising administering a sufficient amount of the composition of claim 30 to a human or animal to prevent or diminish said damage.

57. A method for producing a fiber-treatment composition for the prevention or diminution of photochemical damage caused by insolation, UVA radiation, UVB radiation, X-radiation or gamma radiation in humans or animals by preparing a solution comprising 0.001 to 30% of an α-lipoic acid component based on the total weight of the solution and having a pH of between 0.0 and 6.5.

58. The method of claim 57 through the action of the solution on the fiber material and subsequent removal of the solvent, particularly preferably by drying or spinning.

59. The method of claim 57, on fibers composed of cotton, linen, live wool, wool, natural silk, keratin, synthetic fibers or mixtures thereof.

60. The use of claim 58, on fibers composed of cotton, linen, live wool, wool, natural silk, keratin, synthetic fibers or mixtures thereof.

61. The aqueous solution of claim 30 suitable for pharmaceutical or dietary use.

62. The solution of claim 30, wherein said salt is a creatine, Na, K, NH₄⁺ or ornithine lipoate.

63. The solution of clam 35, wherein said proportion is between 0.2 and 5.0% by weight.

64. The solution of claim 36 having a pH between 2.5 and 4.0.

65. The solution of claim 37, wherein said pharmaceutical excipient or formulation is selected from the group consisting of ethanol, a liquid polyethylene glycol (PEG), propylene glycol, butylene glycol, tetraglycol, benzyl alcohol, sorbitol, mannitol or glycerol.

66. The method of claim 43, wherein said organic cation is an open-chain or cyclic ammonium compound.

67. The method of claim 66, wherein said organic cation is selected from the group consisting of benzylammonium, diisopropylammonium, triethylammonium and cyclohexylammonium.

68. The method of claim 38, wherein said base comprising a complex cation metallic central atom.

69. The method of claim 68, wherein said complex cation comprising iron(III), chromium(III) or cobalt(II).

70. The method of claim 69, wherein said, central metal atom is selected from the group consisting of iron(III), chromium(III) or cobalt(II)

71. The method of claim 69, wherein said base comprises a neutral, cationic or anionic ligand.

72. The method of claim 71, wherein said ligand is selected from the group consisting of water, ammonia, carbonyl, cyano and nitroso, or oxo cation.

73. The method of clam 69, wherein said complex cation comprises an oxovanadium(V) (VO³⁺), oxovanadium (IV) (VO²⁺) or mixtures thereof

74. The method of claim 31, wherein said complex cation comprising iron(III), chromium(III) or cobalt(II).

75. The method of claim 44, wherein said base is employed in stage (a) in an amount of from 0.1-5 .0 mole equivalents.

76. The method of claim 44, wherein said base is employed in an amount of from 0.6 to 1.5 mole equivalents.

77. The method of claim 45 wherein said temperature is from 0 to 50° C.

78. The method of claim 45, wherein said temperature is from 4 to 30° C.

79. The method of claim 46, wherein the pH of the solution at the end of step (a) is between 6.5 to 10.5.

80. The method of claim 48 wherein said temperature is in the range of from 0 to 50° C.

81. The method of claim 48, wherein said temperature is in the range of 4 to 30° C.

82. The method of claim 49, wherein said aid is selected from the group consisting of in particular acetic acid, hydrochloric acid, ascorbic acid and glutamic acid, is used.

83. The method of claim 50, wherein said Lewis acid is selected from the group consisting of carbon dioxide, Ca²⁺ and Fe²⁺.

84. The method of claim 52, wherein said acid is selected from the group consisting of polyphosphoric acid, heptamolybdic acid and dodecatungstophosphoric acid.

85. The method of claim 53, wherein said acid is employed in an, amount of from 0.2 to 4.0 mole equivalents.

86. The method of claim 53, wherein said acid is employed in an amount of from 0.9 to 2.0 mole equivalents.

87. The aqueous solution of claim 30, wherein said aqueous solution is stable for a period of at least one year at a temperature of 15 to 40° C. at a pH of from 2.0 to 5.5.

88. The method of claim 51, wherein said acid is hexaaquoaluminum (III) [(Al(H₂O)₆³⁺].
